(19) United States
(12) Patent Application Publication
(10) Pub. No.: US 2026/0344554 A1
(43) Pub. Date: Nov. 5, 2026

(54) SEED TREATMENT TO INDUCE BACTERIAL BIOFILM FORMATION

(71) Applicant: The Regents of the University of California, Oakland, CA (US)

(72) Inventors: Eduardo BLUMWALD, Davis, CA (US); Dawei YAN, Davis, CA (US)

(21) Appl. No.: 19/118,143

(22) PCT Filed: Oct. 3, 2023
(86) PCT No.: PCT/US2023/034398

§ 371 Date: Apr. 3, 2025

Related U.S. Application Data
(60) Provisional application No. 63/413,187, filed on Oct. 4, 2022.

(51) Int. Cl. A01C 1/06; A01H 3/04

(52) U.S. Cl. CPC A01C 1/06; A01H 3/04

(57) ABSTRACT

The present disclosure provides compositions and methods for producing a crop plant or seeds of the crop plant that induce biofilm formation (e.g., biofilm comprising nitrogen-fixing bacteria), in which the crop plant or the seeds are treated with a compound of Table 1 (e.g., tannic acid).

### CROSS-REFERENCE TO RELATED APPLICATION

This application claims priority to U.S. Provisional Application No. 63/413,187, filed Oct. 4, 2022, the disclosure of which is hereby incorporated by reference in its entirety for all purposes.

### BACKGROUND OF THE INVENTION

Biofilms are essential for optimal colonization of host plant and contribute to nitrogen fixation. Biofilms are often seeded by “aggregates” that are embedded in a self-produced matrix of extracellular polymeric substances (EPS) containing polysaccharides, proteins, lipids, and extracellular DNA. The matrix provides shelter and nutrients for the bacteria, and it contributes to tolerance/resistance toward antimicrobial compounds. In addition, biofilms enable effective interactions by chemical communication (quorum sensing) to remodel the soil bacterial community dynamically, making biofilms one of the most successful modes of life on earth. In some cases, biofilm formation is indispensable for a successful bacterial colonization.

The formation of bacterial biofilms also generates heterogeneity, including the establishment of stable gradients of nutrients, pH, and redox conditions. More importantly, because of the decreased oxygen diffusion across bacterial biofilms, free-living nitrogen-fixing bacteria (Azospirillum brasilens, Pseudomonas stutzeri, etc) are able to fix nitrogen under natural aerobic conditions, since the bacterial nitrogenase is protected from oxygen-induced damage due to the low oxygen concentration at the bacterial surface. Increased biofilm formation can allow the enhanced interaction of the plant roots with the nitrogen-fixing bacteria, allowing nitrogen uptake by the plant and efficient growth even in the presence of reduced inorganic nitrogen in the soil.

### BRIEF SUMMARY OF THE INVENTION

In one aspect, the present disclosure provides a composition comprising a crop plant or seeds of a crop plant treated with a compound of Table 1 (e.g., tannic acid), or a salt thereof, in an amount sufficient to induce biofilm formation, in which the biofilm comprises nitrogen-fixing bacteria.

In some embodiments, the crop plant is a seedling. In certain embodiments, the roots of the crop plant is treated with the compound of Table 1.

In some embodiments, the crop plant is a cereal crop, such as corn, wheat, rice, soy, cotton, canola, and sugarcane. In certain embodiments, the crop plant is rice.

In another aspect, the present disclosure provides a method of producing a crop plant or seeds of the crop plant that induce biofilm formation, in which the biofilm comprises nitrogen-fixing bacteria, the method comprises treating the crop plant or the seeds with a compound of Table 1 (e.g., tannic acid). In some embodiments, the compound is apigenin. In some embodiments, the compound is tannic acid. In some embodiments, the compound is curcumin.

In some embodiments of the method, the method comprises treating the seeds with the compound of Table 1 (e.g., tannic acid) and the treating is performed prior to planting the seeds in a soil comprising the nitrogen-fixing bacteria. In some embodiments, the method further comprises planting the treated seeds in the soil. In some embodiments of the method, the method comprises treating the seeds with the compound of Table 1 (e.g., tannic acid) and the treating is performed after planting the seeds in a soil comprising the nitrogen-fixing bacteria.

In some embodiments of the method, the method comprises treating a crop plant with the compound of Table 1 (e.g., tannic acid). In certain embodiments, the crop plant is a seedling and the treating is performed prior to planting the seedling in a soil comprising the nitrogen-fixing bacteria. In some embodiments, the method further comprises planting the treated seedling in the soil.

In some embodiments, the seeds are treated with the compound by seed coating or seed injection. In certain embodiments, the seed coating is selected from the group consisting of seed dressing, film coating, pelleting, and encrusting.

In some embodiments of the method described herein, the crop plant is a cereal crop, e.g., corn, wheat, rice, soy, cotton, canola, and sugarcane. In certain embodiments, the crop plant is nice.

In some embodiments of the method, the crop plant or the seeds are planted under reduced inorganic nitrogen conditions. In particular embodiments, the amount of inorganic nitrogen in a soil is less than 90%, 80%, 70%, 60% or 50% of the standard amount of nitrogen for the crop plant. In certain embodiments, the nitrogen-fixing bacteria in a soil in which the crop plant or the seeds are grown show greater biofilm formation than control nitrogen-fixing bacteria in a soil in which a control plant not treated with the compound are grown.

In some embodiments, the crop plant grown in a soil comprising the reduced amount of inorganic nitrogen assimilates at least twice the amount of atmospheric nitrogen than the amount assimilated by a control plant grown in equivalent soil, and wherein the seeds of the control plant are not treated with the compound.

In some embodiments, the crop plant or seeds of the crop plant induces a larger area of biofilm than a biofilm induced by a control crop plant or control seeds not treated with the compound of Table 1. In other embodiments, the biofilm contains a greater quantity of nitrogen-fixing bacteria than a biofilm induced by a control crop plant or control seeds not treated with the compound of Table 1. In yet other embodiments, the biofilm produces more fixed nitrogen than a biofilm induced by a control crop plant or control seeds not treated with the compound of Table 1.

In another aspect, the disclosure provides a method for selecting for a compound that induces biofilm formation, the method comprises: 1) treating the crop plant or the seeds with a compound of Table 1: 2) comparing the amount of biofilm formation in the soil in which the crop plant or the seeds are grown with the amount of biofilm formation in the soil in which a control plant or control seeds not treated with a compound of Table 1 are grown; 3) selecting a compound for the crop plant or the seeds that induces more biofilm formation compared to that of the control plant or control seeds, wherein the biofilm comprises nitrogen-fixing bacteria.

In some embodiments, in step 3), the biofilm induced by compound contains a greater quantity of nitrogen-fixing bacteria than that of the biofilm in the soil of the control crop plant or control seeds not treated with the compound of Table 1.

In another aspect, the disclosure provides a method for selecting for a crop plant or seeds of the crop plant that induce greater nitrogen assimilation relative to a control plant or control seeds, the method comprises: 1) treating the crop plant or the seeds with a compound of Table 1; 2) comparing the amount of nitrogen assimilated into the crop plant or the seeds with the amount of nitrogen assimilated into the control crop plant or control seeds not treated with a compound of Table 1; 3) selecting for the crop plant or the seeds that have greater nitrogen assimilation compared to that of the control plant or control seeds.

In some embodiments, the crop plant or the seeds have at least 0.1-fold (e.g., about 0.1, 0.2, 0.3, 0.4, 0.5, 0.6, 0.7, 0.8, 0.9, 1-fold. 2-fold, 3-fold, 4-fold, or more) greater amount of nitrogen assimilated than the amount of nitrogen assimilated into the control crop plant or control seeds not treated with the compound of Table 1.

In another aspect, the disclosure provides a method for selecting for a crop plant that have a greater number of tillers, tassels, and/or spikes, and/or greater seed yield relative to a control plant, the method comprises: 1) treating the crop plant with a compound of Table 1; 2) comparing the number of tillers, tassels, and/or spikes, and/or amount of seed yield with that of the control crop plant not treated with a compound of Table 1; 3) selecting for the crop plant that has the greater number of tillers, tassels, and/or spikes, and/or greater amount of seed yield compared to that of the control plant.

In some embodiments, the crop plant has at least 5% more tillers, tassels, and/or spikes, and/or seed yield than that of the control crop plant not treated with the compound of Table 1.

In some embodiments of the above described methods, the crop plants or seeds are grown under low nitrogen condition. In certain embodiments, the crop plant is selected from the group consisting of corn, wheat, rice, soy, cotton, canola, and sugarcane.

### BRIEF DESCRIPTION OF THE DRAWINGS

FIG. 1. Workflow for chemical screening for biofilm inducers in nitrogen-fixing bacteria.

FIG. 2. Heat map of 2800 compounds on biofilm formation.

FIG. 3. Chemical structures and hierarchical clustering of the top 30 biofilm inducers.

FIG. 4. Hit verification of the biofilm induction effect on two nitrogen-fixing bacteria.

FIG. 5. Apigenin, tannic acid, and curcumin are general biofilm inducers of soil diazotrophs.

FIG. 6A. Veen diagram showing the overlap of the soil diazotrophs whose biofilm can be induced by both apigenin and tannic acid.

FIG. 6B. Veen diagram showing the overlap of the soil diazotrophs whose biofilm can be induced by both apigenin and curcumin.

FIG. 6C. Veen diagram showing the overlap of the soil diazotrophs whose biofilm can be induced by both apigenin, tannic acid, and curcumin.

FIG. 7A. Representative picture of wheat plants grown in the presence of 100% N2 or 30% N2 and in the presence of added apigenin, tannic acid, or curcumin (2 mL of 100 μM).

FIG. 7B. Quantifications of grain yields of plants shown in FIG. 7A.

### DETAILED DESCRIPTION

### Introduction

The disclosure provides compositions and methods for treating a crop plant (e.g., a seedling) or seeds of the crop plant with a compound of Table 1 (e.g., tannic acid), such that once the crop plant or the seeds are planted, the compound would diffuse into the soil and induce the formation of biofilms in nitrogen-fixing soil bacteria. The formed biofilms in turn protect the bacterial nitrogenase enzyme from the damaging effects of oxygen present in the soil and elicit its nitrogen fixing activity with concomitant production of ammonium, which can readily be taken by the crop plant. The increased production of ammonium and uptake by the plants can reduce the use of inorganic nitrogen fertilizers, bringing not only a reduction in the production costs of the grain, but also reducing the deleterious effects of inorganic nitrogen fertilizer to the environment.

### DEFINITIONS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X. 1.02X. 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The terms “crop plant” and “crop as used herein refer to a plant that can be grown and harvested extensively for subsistence and/or profit. A crop plant can generally be cultivated at one place on a large scale. Many crop plants are cultivated in agriculture or aquaculture. Generally crop plants are harvested as food for humans or fodder for livestock.

The term “biofilm” as used herein refers to an accumulation of organisms on a surface (e.g., bacteria, archaea, fungi, molds, algae, or protozoa). A mature biofilm can comprise a colony of microorganisms resident upon a surface. In the present disclosure, biofilm formation refers to the biofilm formed from nitrogen-fixing bacteria.

Plant and/or Seed Treatment

A plant (e.g., a crop plant) or seeds can be treated with a compound of Table 1 (e.g., tannic acid), or a salt thereof. In certain embodiments, the compound is a compound of Table 2. In particular embodiments, the compound is selected from the group consisting of kaempferol, tannic acid, phytol, γ-tocopherol, α-tocopherol, curcumin, orotic acid, and apigenin. In certain embodiments, the compound is tannic acid. In certain embodiments, the compound is apigenin. In certain embodiments, the compound is curcumin. In some embodiments, the plant being treated is a seedling. For example, the roots of the plant (e.g., a seedling) can be treated with a compound of Table 1 (e.g., tannic acid) prior to the plant being planted into the soil containing nitrogen-fixing bacteria. In another example, the roots of the plant (e.g., a seedling) can be treated with a compound of Table 1 (e.g., tannic acid) after the plant being planted into the soil containing nitrogen-fixing bacteria. In some embodiments, the compound (e.g., tannic acid) can be injected into the roots of the plant. In other embodiments, the roots of the plant can be submerged in a solution containing the compound (e.g., tannic acid) such that a sufficient amount of the compound remains in the roots to induce biofilm formation that contains nitrogen-fixing bacteria. In further embodiments, the plant (e.g., a crop plant) or seeds that are treated with a compound of Table 1 (e.g., tannic acid) can also be treated with diazotrophs such as rhizobia.

A plant (e.g., a crop plant) or seeds can be treated with at least 0.1 mM of a compound of Table 1 (e.g., tannic acid). In some embodiments, a plant (e.g., a crop plant) or seeds can be treated with between 0.1 mM and 500 mM (e.g., between 1 mM and 500 mM, between 5 mM and 500 mM, between 10 mM and 500 mM, between 20 mM and 500 mM, between 40 mM and 500 mM, between 60 mM and 500 mM, between 80 mM and 500 mM, between 100 mM and 500 mM, between 150 mM and 500 mM, between 200 mM and 500 mM, between 250 mM and 500 mM, between 300 mM and 500 mM, between 350 mM and 500 mM, between 400 mM and 500 mM, between 450 mM and 500 mM, between 0.1 mM and 450 mM, between 0.1 mM and 400 mM, between 0.1 mM and 350 mM, between 0.1 mM and 300 mM, between 0.1 mM and 250 mM, between 0.1 mM and 200 mM, between 0.1 mM and 150 mM, between 0.1 mM and 100 mM, between 0.1 mM and 80 mM, between 0.1 mM and 60 mM, between 0.1 mM and 40 mM, between 0.1 mM and 20 mM, between 0.1 mM and 10 mM, between 0.1 mM and 5 mM, or between 0.1 mM and 1 mM) of a compound of Table 1 (e.g., tannic acid). In some embodiments, the treated plants (e.g., seedlings) or seeds (e.g., plants or seeds coated with a compound of Table 1, e.g., tannic acid) contain at least 0.01 mM of a compound of Table 1 (e.g., tannic acid). In some embodiments, the treated plants (e.g., seedlings) or seeds (e.g., plants or seeds coated with a compound of Table 1, e.g., tannic acid) contain between 0.01 mM and 100 mM (e.g., between 0.05 mM and 100 mM, between 0.1 mM and 100 mM, between 0.2 mM and 100 mM, between 0.4 mM and 100 mM, between 0.6 mM and 100 mM, between 0.8 mM and 100 mM, between 1 mM and 100 mM, between 5 mM and 100 mM, between 10 mM and 100 mM, between 15 mM and 100 mM, between 20 mM and 100 mM, between 25 mM and 100 mM, between 30 mM and 100 mM, between 35 mM and 100 mM, between 40 mM and 100 mM, between 50 mM and 100 mM, between 60 mM and 100 mM, between 70 mM and 100 mM, between 80 mM and 100 mM, between 90 mM and 100 mM, between 0.01 mM and 90 mM, between 0.01 mM and 80 mM, between 0.01 mM and 70 mM, between 0.01 mM and 60 mM, between 0.01 mM and 50 mM, between 0.01 mM and 40 mM, between 0.01 mM and 30 mM, between 0.01 mM and 20 mM, between 0.01 mM and 10 mM, between 0.01 mM and 8 mM, between 0.01 mM and 6 mM, between 0.01 mM and 4 mM, between 0.01 mM and 2 mM, or between 0.01 mM and 1 mM) of a compound of Table 1 (e.g., tannic acid). The amount or concentration of the compound (e.g., tannic acid) used to treat the plants or seeds, or the amount or concentration of the compound (e.g., tannic acid) that remains on the surface of the plants or seeds after treatment, will depend on the type of compound used, the compound's solubility, the growth rate of the plants or seeds, as well as the type of soil. In some embodiments, the compound (e.g., tannic acid) on the surface of the plants or seeds can diffuse into the soil such that the soil can have a concentration of the compound (e.g., tannic acid) that is at least 1 μM. e.g., between 1 and 10 μM (e.g., 1, 2, 3, 4, 5, 6, 7, 8, 9, or 10 μM).

A compound of Table 1 (e.g., tannic acid) can be incorporated into seeds of a plant (e.g., a crop plant) using various seed treatment techniques. In some embodiments, seeds of the plant can be coated with the compound (e.g., tannic acid). In some embodiments, the compound (e.g., tannic acid) can be injected into the seeds.

Examples of techniques that can be used to coat seeds with a compound of Table 1 include, but are not limited to, seed dressing, film coating, pelleting, and encrusting. Seed coating is the process of applying exogenous materials to the surface of the seeds. In some embodiments, seed coating is used to modify the physical properties of seed and for the delivery of active ingredients (e.g., tannic acid). In some embodiments, one or more compounds of Table 1 (e.g., tannic acid) can be applied to the surface of the seeds with the help of a binder and in some embodiments, a filler can act as a carrier. Seed coating can vary from simple on-farm applications to sophisticated and industrialized procedures. Although the processes used by farmers and industrial companies may differ, the principle is basically the same. Overall, it includes, seeds inside a container (e.g., rotating drum, cement mixer), where a binder (e.g., adhesive compound), a filler (bulking agent) if needed, and active ingredients (e.g., tannic acid) are mixed. Fillers can be single or mixed components, and the most commonly applied are peat, talc, and lime. These components can function as compound carriers and in some cases, modify seed size, shape, and/or weight. Some ingredients like alginate can be used both as filler and binder. In some cases, biochar and chitosan have been also considered as fillers/carriers for seed coating. Binders, natural or synthetic polymers such as methyl cellulose, carboxymethyl cellulose, gum arabic, or polysaccharide Pelgel can be generally added during or toward the end of the coating process in order to bind the exogenous materials (e.g., tannic acid) and reduce the amount of dust in the final product. Some adhesives (e.g., gum arabic and xanthan gum) can also be used. The selection of the proper type and concentration of binder and filler can be crucial for seed germination and plant development.

The classification of seed coating types is usually based on the weight, size, and grouping properties of the seeds. Types of seed coating include, but are not limited to, seed dressing, film coating, pelleting, and encrusting. Seed dressing, which refers to the application of finely milled solids dusted onto the surface of seeds in small amounts. Film coating includes application of a thin layer of external material with little change of the seed shape, size, and weight. In some embodiments of film coating, a solution or suspension is applied onto the seeds. In some embodiments, film coating allows better treatment precision and minimizes the production of dust. In comparison with other seed coating types, in some embodiments, film coating has a lower interference with seed germination and a prompter release of active components.

Seed pelleting comprises fillers and liquid binders applied to the seeds that may cause a significant increase in weight and volume of the seeds. Pelleting usually modifies seed morphology into a spherical or ovoid shape. If the original seed shape is still maintained, the term used for this type of seed coating is seed encrusting. Pelleting and encrusting can increase the amount of applied active ingredients and improve seed handling and sowing, especially for irregularly shaped seeds.

Depending on the type of coating, specific equipment is considered. The rotating pan is the most common device used for seed coating (e.g., pelleting, encrusting, dressing, and film coating). It usually consists of an inclined round pan rotating in slow motion, where materials are gradually added, followed by size sorting (sieving and screening) and then drying. Film coating and encrusting can also be carried out using a fluidized or spouted bed, a cylindrical apparatus where seeds are kept in suspension by a constant vertical/bottom-up hot airflow, while being sprayed with coating materials. The warm airflow allows moisture evaporation. Another device used for most seed coating types is the rotary coater or rotor-stator, a cylindrical drum with two rotating base disks, a concave one, whose rotation causes seeds to move steadily along the drum walls, and a smaller one that allows the atomization and projection of liquid/slurry coating to the rotating seed mass.

Additional descriptions of seed coating, as well as materials and equipment needed can be found in, e.g., Pedrini, et al., Trends Plant Sci. 22(2):106-116, 2017; Rocha et al., Front Plant Sci. 10:1357, 2019; Ma, Biotechnol Adv 37(7):107423, 2019; and Ehsanfar and Modarres-Sanavy, Commun Agric Appl Biol Sci. 70(3):225-9, 2005.

In some embodiments, the treated plants (e.g., seedlings) or seeds (e.g., plants or seeds coated with a compound of Table 1. e.g., tannic acid) are not in the soil. In some embodiments, the treated plants (e.g., seedlings) or seeds (e.g., plants or seeds coated with a compound of Table 1, e.g., tannic acid) are in a container, e.g., a bag, a box, etc. In some embodiments, the treated plants (e.g., seedlings) or seeds (e.g., plants or seeds coated with a compound of Table 1, e.g., tannic acid) contain an amount of the compound of Table 1, e.g., tannic acid, that is higher than the amount of the compound, if any, in the soil. In some embodiments, the treated plants (e.g., seedlings) or seeds (e.g., plants or seeds coated with a compound of Table 1, e.g., tannic acid) contain an amount of the compound of Table 1, e.g., tannic acid, that is higher than the amount of the compound, if any, in the soil, and the treated plants (e.g., seedlings) or seeds are not in the soil.

In some embodiments, in addition to treating (e.g., coating) the seeds with a compound of Table 1 (e.g., tannic acid), the seeds can also be treated with nitrogen-fixing bacteria, e.g., Glucanoacetobacter diazotrophicus. In some embodiments, in addition to treating (e.g., coating) the seeds with a compound of Table 1 (e.g., tannic acid), the seeds can also be treated with diazotrophs, e.g., rhizobia. In some embodiments, in addition to treating (e.g., coating) the seeds with a compound of Table 1 (e.g., tannic acid), the seeds can also be treated with nitrogen-fixing bacteria, e.g., Glucanoacetobacter diazotrophicus, and diazotrophs, e.g., rhizobia.

In other embodiments, a compound of Table 1 (e.g., tannic acid) can be added to a soil containing nitrogen-fixing bacteria, e.g., Glucanoacetobacter diazotrophicus, to induce biofilm formation. In certain embodiments, a compound of Table 1 (e.g., tannic acid) can be added to a soil containing nitrogen-fixing bacteria, e.g., Glucanoacetobacter diazotrophicus, before the plants (e.g., seedlings) or seeds (e.g., plants or seeds coated with a compound of Table 1, e.g., tannic acid) are planted in the soil. In certain embodiments, a compound of Table 1 (e.g., tannic acid) can be added to a soil containing nitrogen-fixing bacteria, e.g., Glucanoacetobacter diazotrophicus, after the plants (e.g., seedlings) or seeds (e.g., plants or seeds coated with a compound of Table 1, e.g., tannic acid) are planted in the soil. In some embodiments, the compound can be added to the soil as a solution containing the desired concentration of the compound. In other embodiments, the compound can be added to the soil as a solid, e.g., in powder form or in pellet form. Whether the compound is added to the soil as a solution or as a solid, the compound is mixed thoroughly and evenly in the soil containing the nitrogen-fixing bacteria. In other embodiments, diazotrophs such as rhizobia can also be added to the soil containing nitrogen-fixing bacteria.

In other embodiments, treatment may be carried out in the form of any kind of soil application, such as in-furrow, by drip application, soil incorporation, drench application, sprinkler irrigation, micro injection, or granule application. In some embodiments, the treatment is carried out in the soil, prior to germination of a seed and/or in the soil in contact with a root of a plant or where a plant is intended to grow. In some embodiments, the treatment is carried out repeatedly. In some embodiments, repeatedly may refer to at least two, at least three, at least four or even at least five treatments prior to sowing/planting and/or during germination and/or growth of the plant.

In some embodiments, the method of the disclosure further comprises applying, simultaneously or sequentially, at least one further plant protection agent, e.g., a nematicide, an insecticide, a bactericide, a miticide, a fungicide or another agent promoting or improving plant health.

### Plants

The present compositions and methods can be used to modify any plant, including monocots and dicots, grains, trees, and vegetable crops, in order to increase its ability to interact with nitrogen-fixing bacteria in the soil. In particular embodiments, the plant is a crop species such as corn, wheat, rice, soy, cotton, canola, or sugarcane. In particular embodiments, the crop plant is a grain crop. Crops that can be used include, but are not limited to, cereals, oilseeds, pulses, hays, and others. A non-limiting list of cereals that can be used includes rice (e.g., Oryza, Zizani spp.), wheat (e.g., Triticum aestivum), barley (e.g., Hordeum vulgare), oat (e.g., Avena sativa), rye (e.g., Secale cereal), triticale (e.g., Triticosecale spp.), corn (e.g., Zea mays), sorghum Sorghum spp., millet (e.g., Digitaria, Echinochloa, Eleusine, Panicum, Setaria, Pennisetum, spp.), canary seed (e.g., Phalaris canariensis), teff (e.g., Eragrostis abyssinica), and Job's Tears (e.g., Coix lacryma-jobi). In particular embodiments, the plant is rice. e.g., Oryza sativa. A non-limiting list of oilseeds includes soybeans (e.g., Glycine spp.), peanuts (e.g., Arachis hypogaea), canola and mustard (e.g., Brassica spp., Brassica napus), sunflower, (e.g., Helianthus annuus), safflower (e.g., Carthamus spp., and flax (e.g., Linum spp.). A non-limiting list of pulses include pinto beans (e.g., Phaseolus vulgaris), lima beans (e.g., Phaseolus lunatus), mungo beans (e.g., Phaseolus mung), adzuki beans (e.g., Phaseolus angularis), chickpeas (e.g., Cicer arietinum), field, green and yellow peas (e.g., Pisum spp.), lentils (e.g., Lens spp.), fava beans (e.g., Vicia faba), and others including Dolichos, Cajanus, Vigna, Pachyrhizus, Tetragonolobus, spp. A non-limiting list of hay and pasture plants includes grasses such as Meadow Foxtail (e.g., Alopecurus pratensis). Brome (e.g., Bromus spp.), Orchard Grass (e.g., Dactylis glomerata), Fescue (e.g., Festuca spp.), rye grass (e.g., Lolium spp.), reed canary grass (e.g., Phalaris arundinacea), Kentucky blue grass (e.g., Poa pratensis), Timothy (e.g., Phleum pretense), and redtop (e.g., Agropyron spp.), as well as legumes such as alfalfa and yellow trefoil (e.g., Medicago spp., Medicago sativa), clovers (Trifolium spp.), birdsgoot trefoil (e.g., Lotus corniculatus), and vetch (e.g., Vicia spp.). Other plants that can used includes buckwheat, tobacco, hemp, sugar beets, and amaranth. In some embodiments, the plant is a shrub such as cotton (e.g., Gossypium hirsutum, Gossypium barbadense.) In some embodiments, the plant is a grass such as sugarcane (e.g., Saccharum officinarum).

In some embodiments, the plant is a tree. Any tree can be modified using the present methods, including angiosperms and gymnosperms. A non-limiting list of trees includes. e.g., cycads, ginkgo, conifers (e.g., araucarias, cedars, cypresses. Douglas firs, firs, hemlocks, junipers, larches, pines, podocarps, redwoods, spruces, yews), monocotyledonous trees (e.g., palms, agaves, aloes, dracaenas, screw pines, yuccas) and dicotyledons (e.g., birches, elms, hollies, magnolias, maples, oaks, poplars, ashes, and willows). In a particular embodiment, the tree is a poplar (e.g., cottonwood, aspen, balsam poplar), e.g., Populus alba, Populus grandidentata, Populus tremula, Populus tremuloides, Populus deltoids, Populus fremontii, Populus nigra, Populus angustifolia, Populus balsamifera, Populus trichocarpa, or Populus heterophylla.

In some embodiments, the plant is a vegetable. Vegetables that can be used include, but are not limited to, Arugula (Eruca sativa), Beet (Beta vulgaris vulgaris), Bok choy (Brassica rapa), Broccoli (Brassica oleracea). Brussels sprouts (Brassica oleracea), Cabbage (Brassica oleracea). Celery (Apium graveolens), Chicory (Cichorium intybus), Chinese mallow (Malva verticillata), Garland Chrysanthemum (Chrysanthemum coronarium), Collard greens (Brassica oleracea), Common purslane (Portulaca oleracea), Coin salad (Valerianella locusta), Cress (Lepidium sativum), Dandelion (Taraxacum officinale), Dill (Anethum graveolens), Endive (Cichorium endivia), Grape (Vitis), Greater plantain (Plantago major). Kale (Brassica oleracea), Lamb's lettuce (Valerianella locusta), Land cress (Barbarea verna), Lettuce (Lactuca sativa), Mustard (Sinapis alba), Napa cabbage (Brassica rapa), New Zealand Spinach (Tetragonia tetragonioides), Pea (Pisum sativum), Poke (Phytolacca Americana), Radicchio (Cichorium intybus), Sorrel (Rumex acetosa), Sour cabbage (Brassica oleracea), Spinach (Spinacia oleracea), Summer purslane (Portulaca oleracea), Swiss chard (Beta vulgaris cicla), Turnip greens (Brassica rapa), Watercress (Nasturtium officinale), Water spinach (Ipomoea aquatic), and Yarrow (Achillea millefolium). Also included are fruits and flowers such as gourds, squashes, Pumpkins, Avocado. Bell pepper, Cucumber, Eggplant, Sweet pepper. Tomato. Vanilla, Zucchini, Artichoke, Broccoli, Caper, and Cauliflower.

### Assessing Biofilm Formation

Any of a number of assays can be used to assess plants or seeds treated with a compound of Table 1 (e.g., tannic acid), or a salt thereof, for the ability to induce biofilm formation. For example, the biofilm produced by the nitrogen-fixing bacteria, e.g., Glucanoacetobacter diazotrophicus, can be assessed, i.e., quantified by incubating the crop plants (e.g., seedlings) or the seeds treated with the compound with the nitrogen-fixing bacteria in the wells of a microtiter plate, removing the cultures from the plate, washing the wells, adding a visualizing solution (e.g., crystal violet), rinsing and drying the plate, and then adding ethanol and measuring absorbance at, e.g., 540 nm.

In some embodiments, nitrogen-fixing bacteria that express a label such as a fluorescent protein (e.g., mCherry) can be used to assess biofilm formation. In some embodiments, the bacteria can also express labeled components of biofilms, e.g., in bacteria transformed with gumDpro::GFP. The double labeling in such bacteria allows the visualization of the bacteria and, independently, the development of biofilm.

The nitrogen-fixing activity of the bacteria can be assessed, e.g., using an acetylene reduction assay (ARA), in which bacteria are cultured in the presence of acetylene gas, and the conversion of acetylene to ethylene measured by, e.g., gas chromatography.

The plants and seeds treated with a compound of Table 1 (e.g., tannic acid) can also be assessed in any of a number of ways. For example, the treated plants or seeds can be grown in the presence of fluorescently labeled nitrogen-fixing bacteria, and the adherence of the bacteria to the plant root hairs, either attached to the root surface or present inside the plant tissues, can be determined. The plants can also be assessed by determining the number of tillers and/or the seed yield. In some embodiments, the assimilation of nitrogen fixed by bacteria in the soil is assessed by, e.g., growing the plants or seeds in the presence of ¹⁵N₂ gas, and then measuring the level of ¹⁵N assimilated in the plant leaves, e.g., using mass spectroscopy.

In some embodiments, plants generated using the present compositions and methods involving plants or seeds treated with a compound of Table 1 (e.g., tannic acid) show an increase of at least 5%, 10%, 15%, 20%, 25%, 30%, 40%, 50%, 60%, or more in the number of tillers/tassels/spikes and/or in the seed yield as compared to plants generated from non-treated plants or seeds. In some embodiments, plants generated using the present compositions and methods involving plants or seeds treated with a compound of Table 1 (e.g., tannic acid) induce an increase of at least about 0.1 (i.e., an increase of about 10%), 0.2, 0.3, 0.4, 0.5, 0.6, 0.7, 0.8, 0.9, 1-fold, 2-fold, 3-fold, 4-fold, or more, in biofilm formation (e.g., biofilm comprising Glucanoacetobacter diazotrophicus or other nitrogen-fixing bacteria) as compared to plants generated from non-treated plants or seeds. In some embodiments, plants generated using the present compositions and methods involving plants or seeds treated with a compound of Table 1 (e.g., tannic acid) induce an increase of at least about 0.1, 0.2, 0.3, 0.4, 0.5, 0.6, 0.7, 0.8, 0.9, 1-fold, 2-fold, 3-fold, 4-fold, or more, of nitrogen assimilation when grown under low nitrogen conditions as compared to plants generated from non-treated plants or seeds.

Because of the increased assimilation of nitrogen-fixing bacteria by plants generated using the present compositions and methods involving plants or seeds treated with a compound of Table 1 (e.g., tannic acid), the present plants can assimilate sufficient nitrogen to produce high yields even when inorganic nitrogen levels in the soil are low. As used herein, “reduced” or “low” or “minimal” inorganic “nitrogen conditions” or “nitrogen levels” refers to conditions in which the level of inorganic nitrogen, e.g., the level resulting from the introduction of fertilizer, is lower than the level that would normally be used for the crop plant, or which is recommended for the crop plant. For example, for rice plants, a level of inorganic nitrogen of less than 50 ppm can be used, e.g. about 25 ppm. In some embodiments, the level of inorganic nitrogen is at least about 10%, 20%, 30%, 40%, 50%, 60%, 70%, or 80% lower than the normal or recommended level. In some embodiments, the plants and seeds treated with a compound of Table 1 (e.g., tannic acid) can be planted in a soil containing a level of inorganic nitrogen that is less than 50 ppm, less than 45 ppm, less than 40 ppm, less than 35 ppm, less than 30 ppm, less than 25 ppm, less than 20 ppm, less than 15 ppm, less than 10 ppm, or less than 5 ppm.

In some embodiments of the methods described herein, the crop plant or seeds of the crop plant can induce a larger area of biofilm (e.g., an area of biofilm that is at least 5%, 10%, 15%, 20%, 25%, 30%, 35%, 40%, 45%, or 50% larger) than a biofilm induced by a control crop plant or control seeds not treated with a compound of Table 1 (e.g., tannic acid). In certain embodiments, the biofilm contains a greater quantity (e.g., a greater number of bacterial cells in the biofilm; e.g., at least 5%, 10%, 15%, 20%, 25%, 30%, 35%, 40%, 45%, or 50% greater) of nitrogen-fixing bacteria than a biofilm induced by a control crop plant or control seeds not treated with a compound of Table 1 (e.g., tannic acid). The biofilm can be quantified by cellular imaging and/or automated cell counting. For example, light and confocal microscopy can be used to count bacterial cells and determine total biofilm volume. Instruments such as automated cell counters and flow cytometers can also be used to quantify biofilms. Other methods and techniques to measure and assay biofilm are described in the art, see. e.g., Wilson et al., Res Rev J Eng Technol. 6(4). 2017.

In some embodiments of the methods described herein, the biofilm induced by the treated plants or seeds described herein can produce more fixed nitrogen than a biofilm induced by a control crop plant or control seeds not treated with a compound of Table 1 (e.g., tannic acid). Methods and techniques to measure nitrogen fixation are available in the art, such as ¹⁵N isotopic dilution, ¹⁵N natural abundance, acetylene reduction assay, microbial bioassay, ureide content measurement, etc. The methods and techniques are described in, e.g., Fonseca-Lopez et al., Cienc. Tecnol. Agropecuaria 21(1), 2020.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

Identifying Chemicals that Induce Biofilm Formation

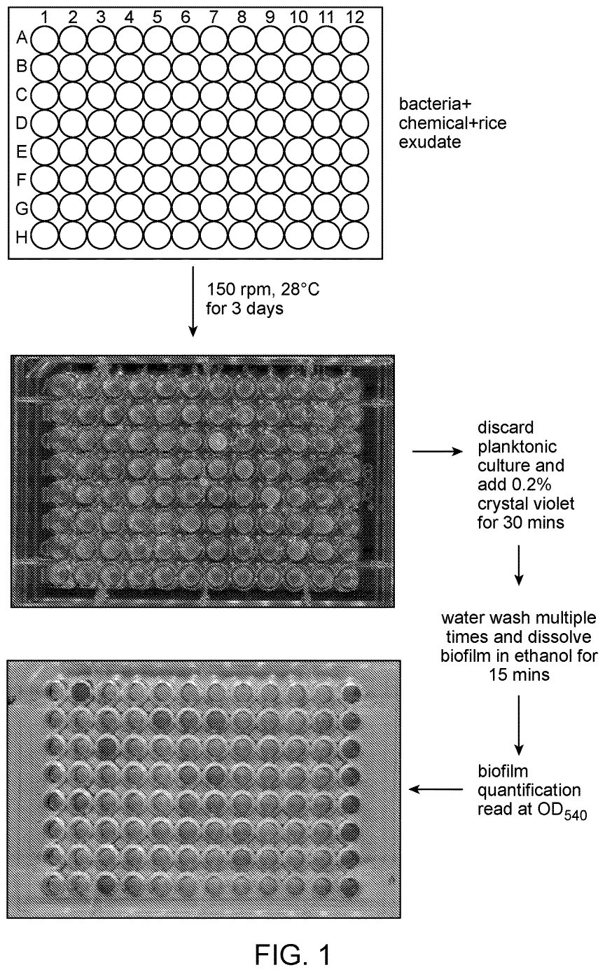

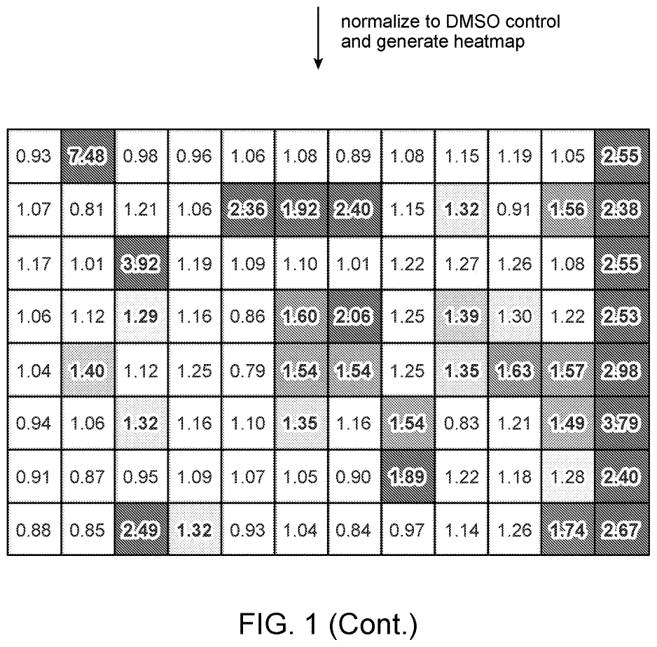

A chemical library screen was carried out to assess the ability of different flavonoids to induce the formation of biofilms in a nitrogen-fixing soil bacteria. The chemical library included 2,800 natural compounds (TargetMol L6000-Natural Compound Library). The bacteria Gluconacetobacter diazotrophicus were used. The workflow of the chemical screening is shown in FIG. 1. Briefly, Gluconacetobacter diazotrophicus cultures were grown overnight in a modified ATCC medium (0.5% yeast extract, 0.3% peptone, 1.5% sucrose, 1.5% mannitol) at 28 °C. The cultures were diluted at 1:50 in fresh ATCC medium and cultures were grown to OD600=0.4. The cultures were pelleted and the supernatant was discarded. The pellets were washed three times with sterile water and then suspended with exudates from 3-day-old Kitaake rice germinating seedlings to a final OD600=0.01.

The exudates used for biofilm assays were collected by germinating 50 Kitaake seeds in 25 ml of sterile milli-Q water for 3 days at 28 °C in the dark. Two μL of the compound (10 mM) from the chemical library was added to 198 μL of bacteria plus Kitaake exudates in each well of a sterile 96 well plate (Corning 3595), resulting in a final concentration of 100 μM of each compound. The 96-well plates were shaken (150 rpm at 28 °C) for 3 days. After incubation, the planktonic cells were discarded. Two hundred L of a crystal violet solution (0.2% crystal violet, 2% ethanol in water) were added to each plate well and the plates were shaken at 28 °C (150 rpm for 30 min). The solution in each well was discarded and the plates were rinsed 3-4 times with water and air-dried. Two hundred μL of 95% ethanol were added to each well to solubilize the crystal violet and shaken at 28 °C (150 rpm for 15 min), and the absorbance was measured at 540 nm (Biotek Synergy Mx plate reader).

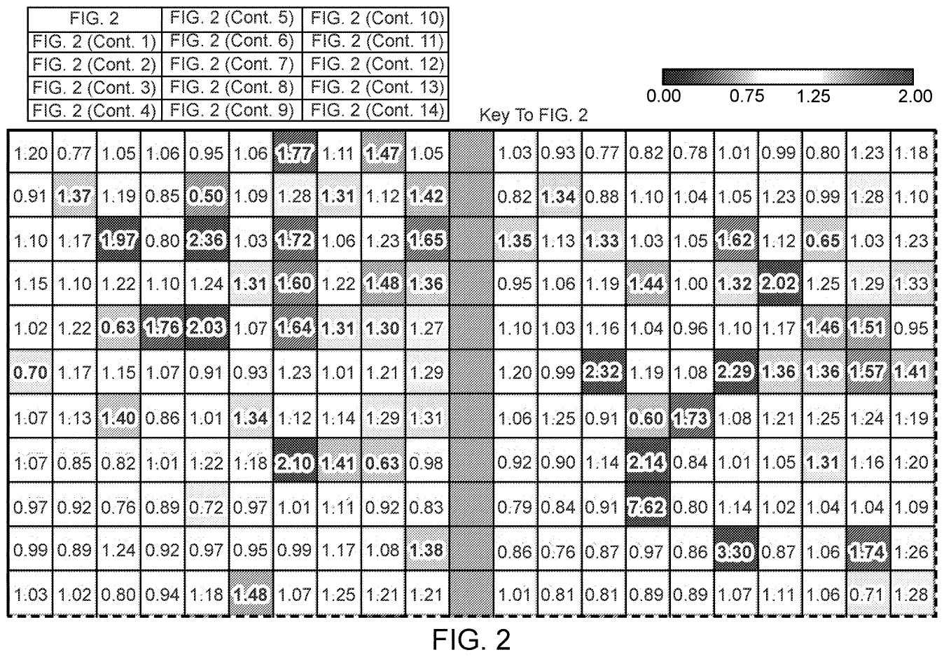

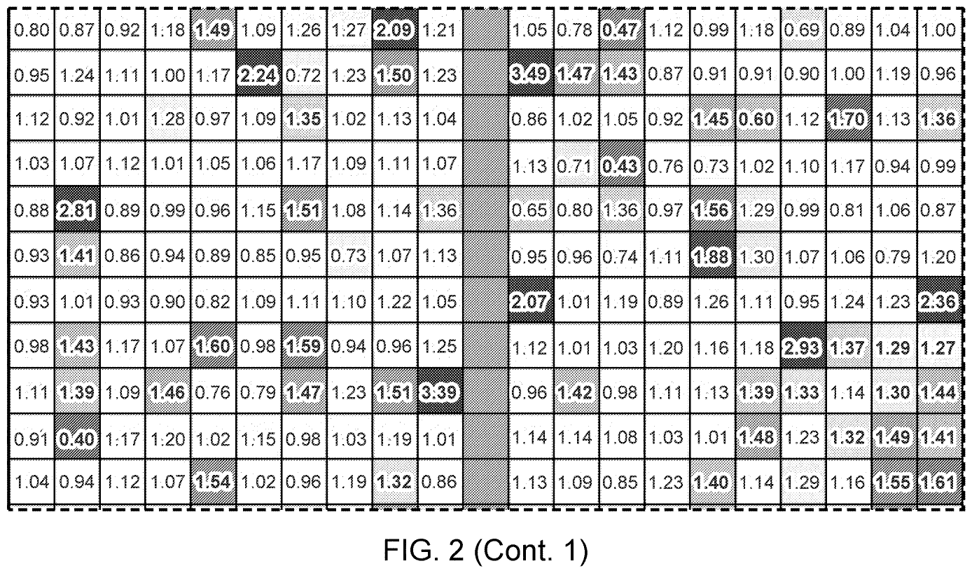

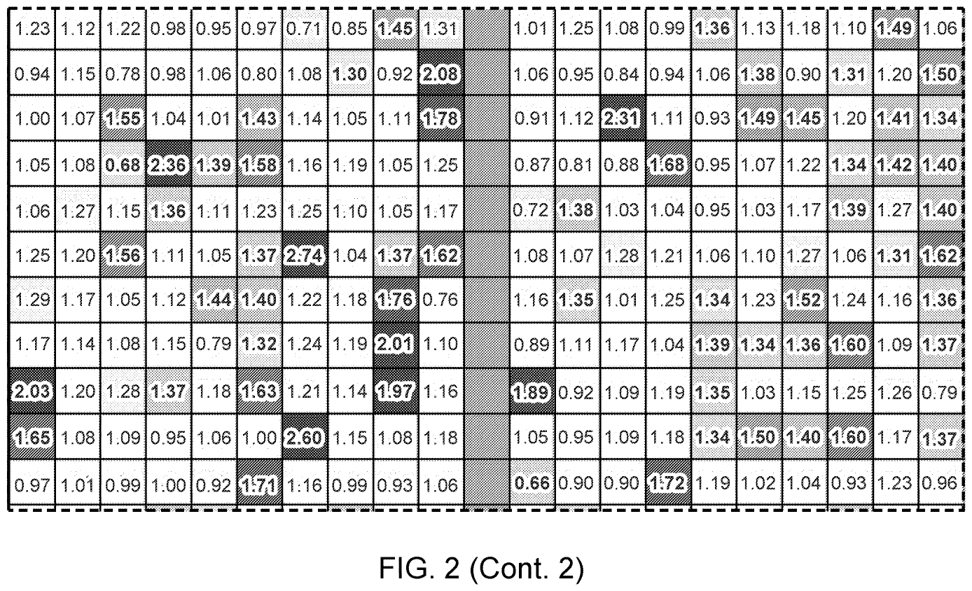

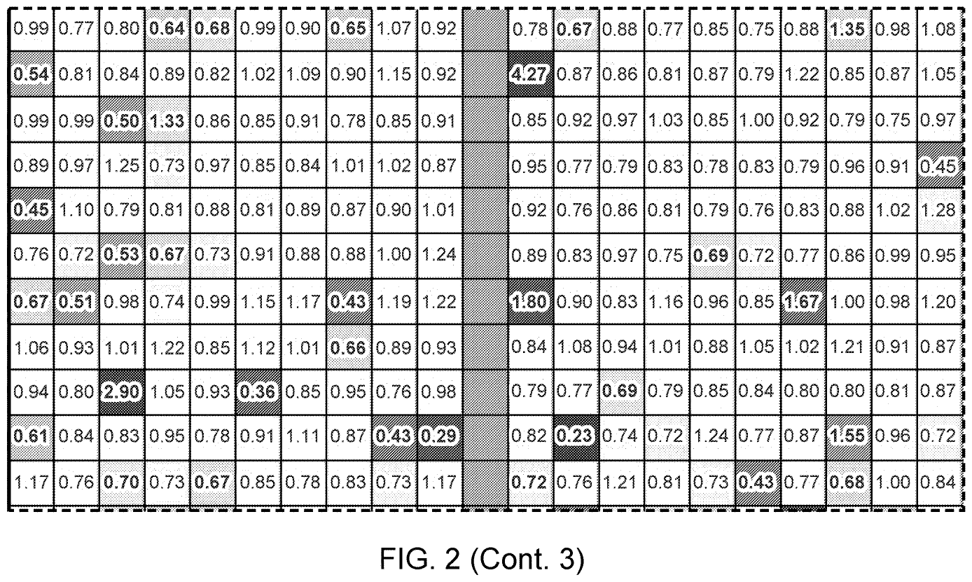

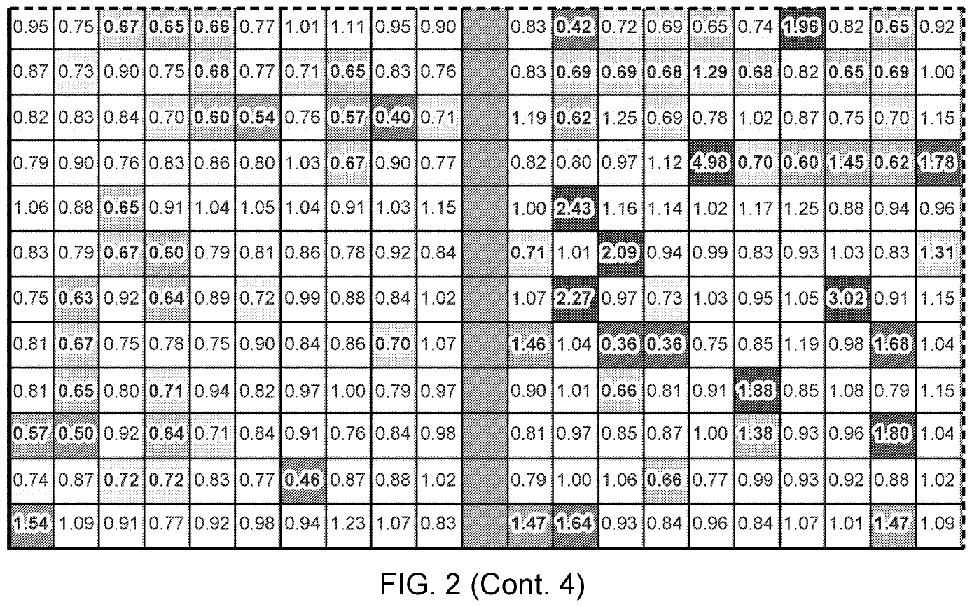

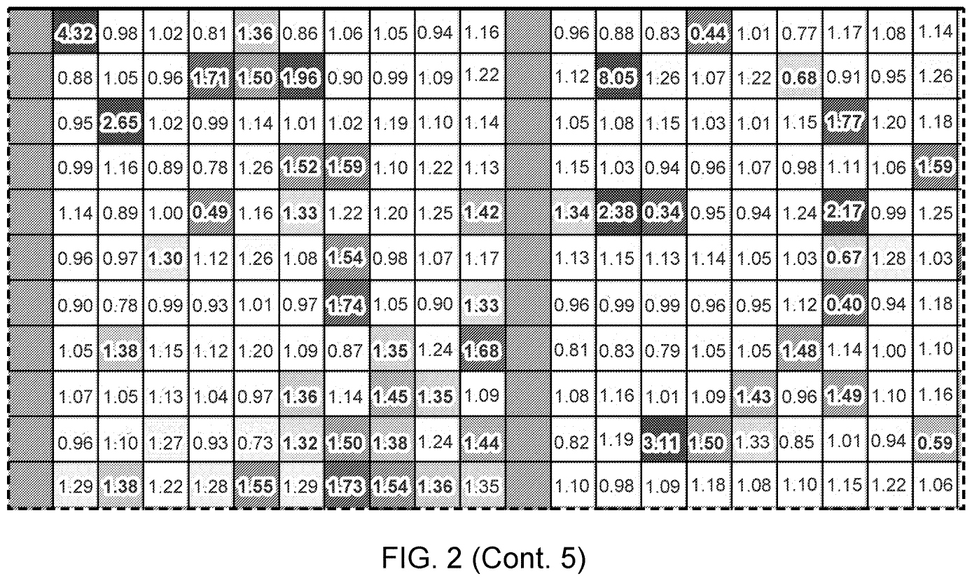

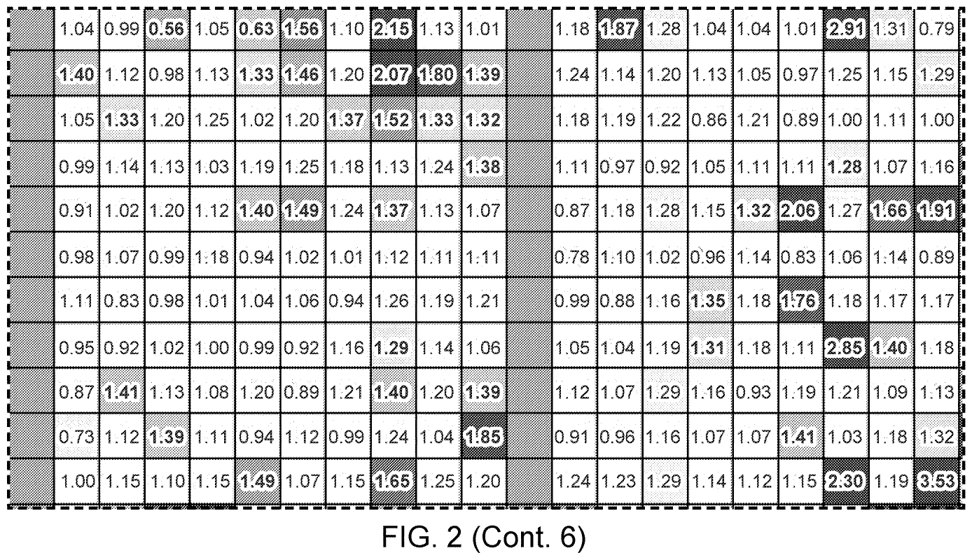

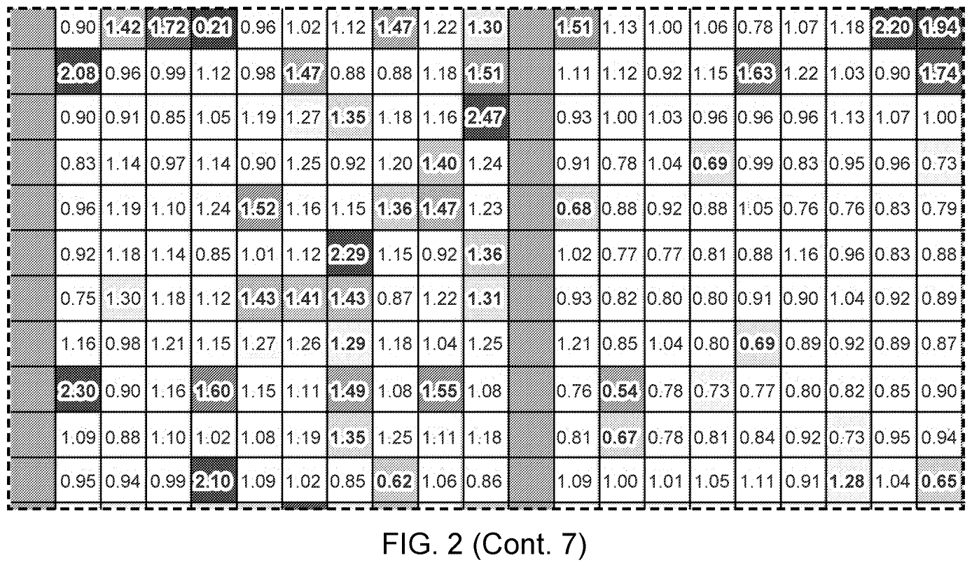

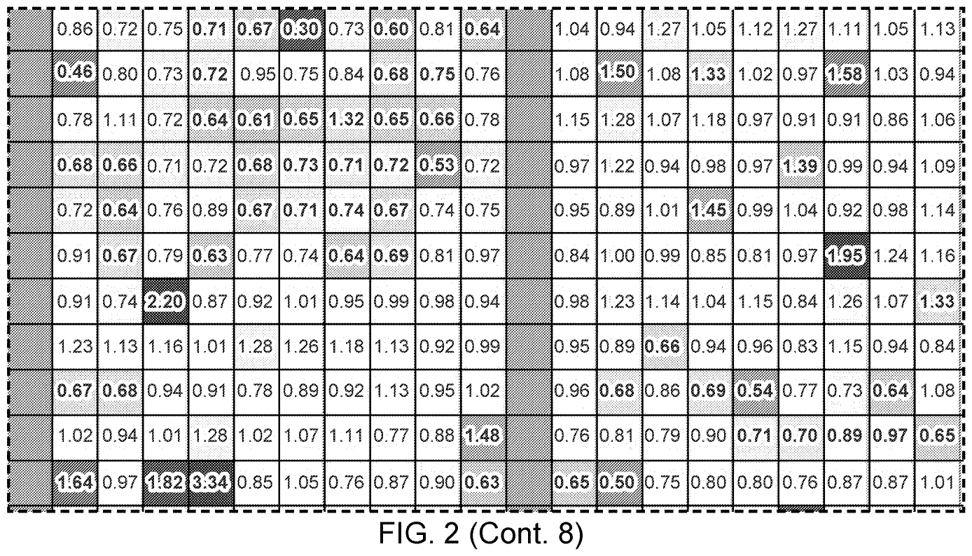

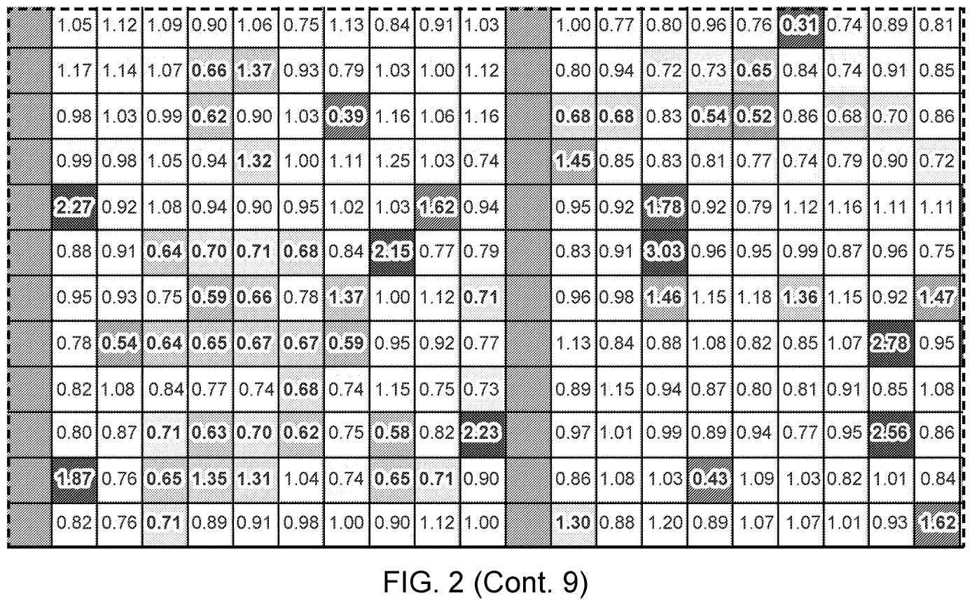

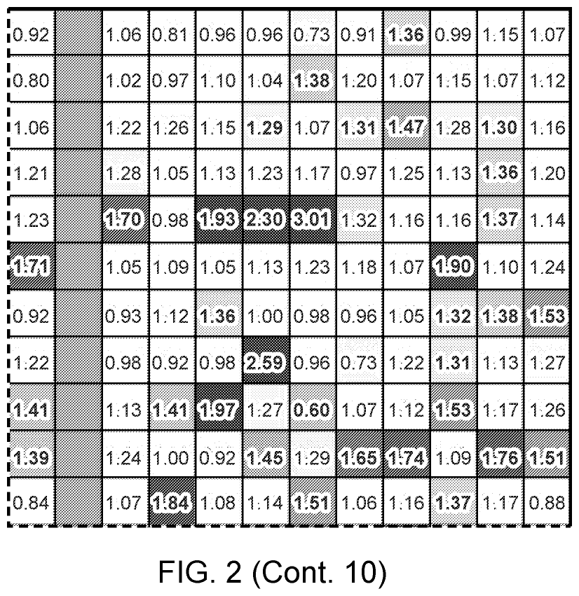

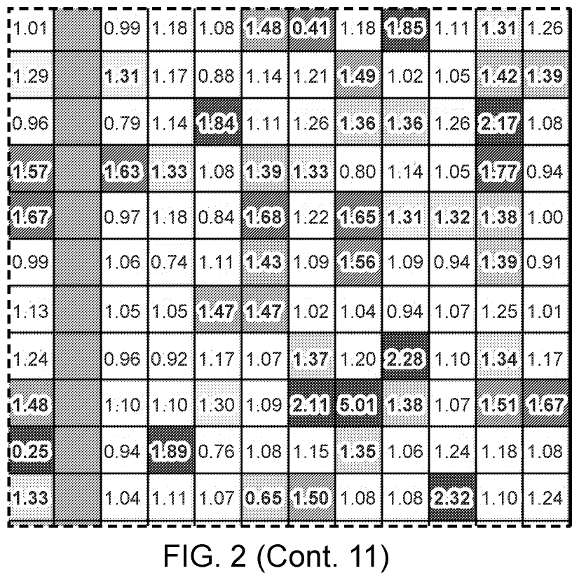

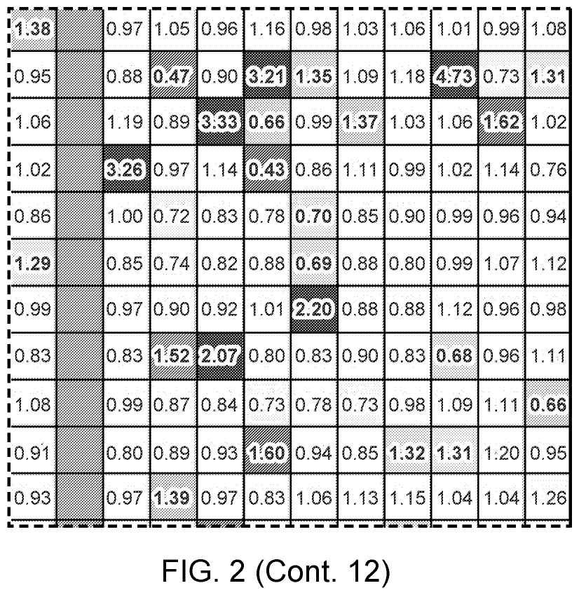

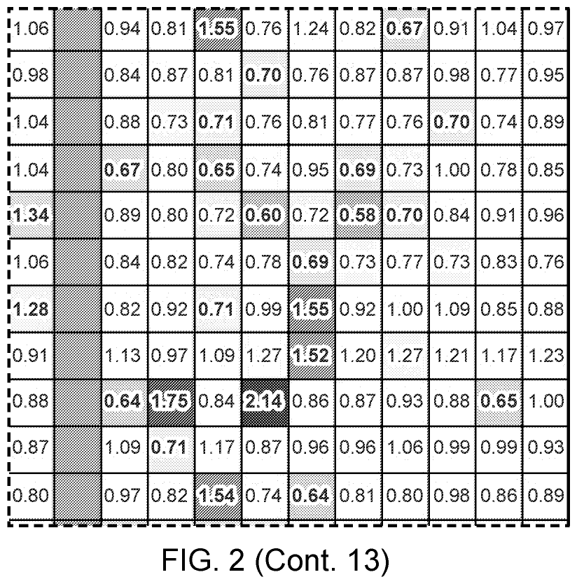

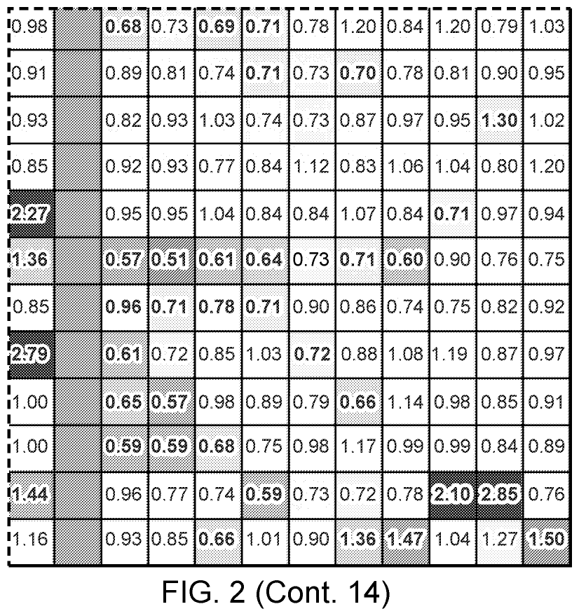

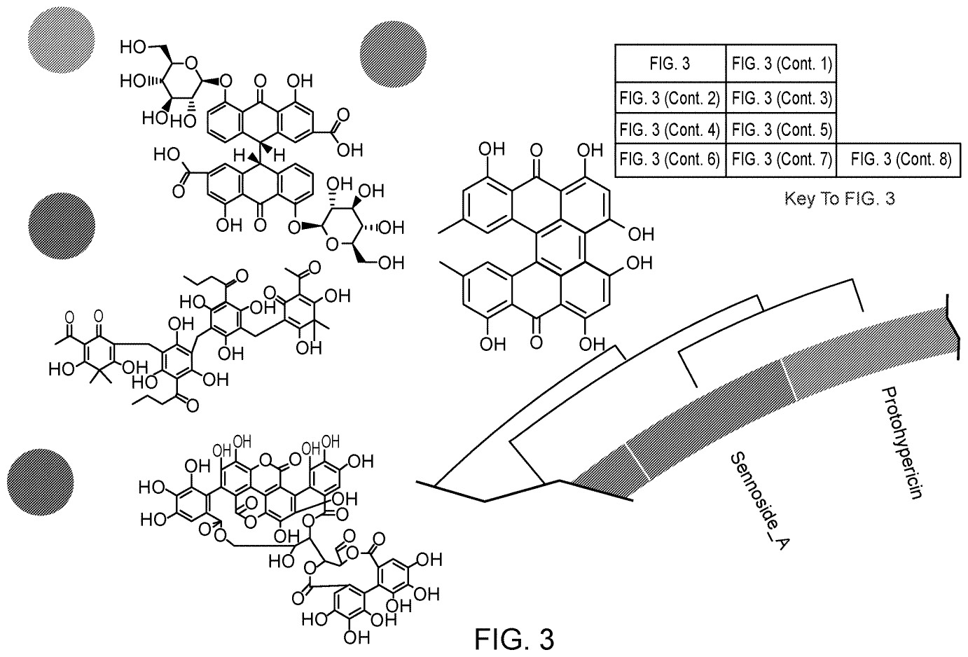

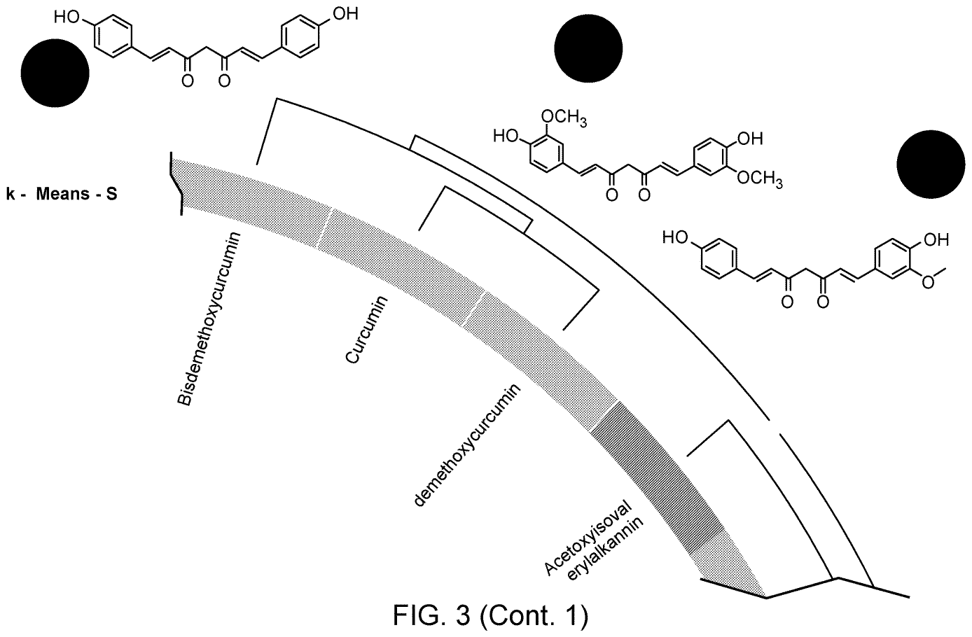

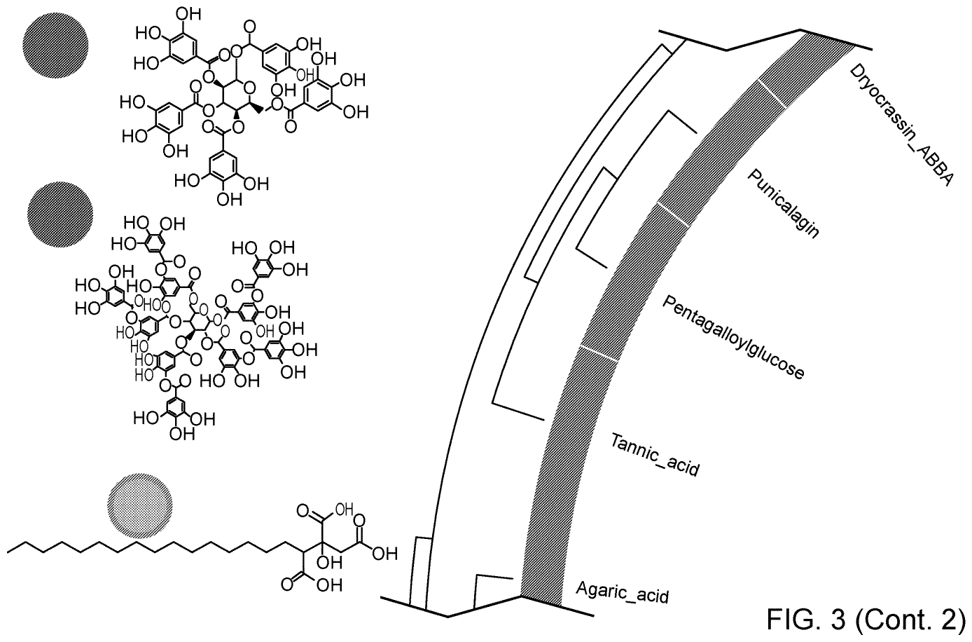

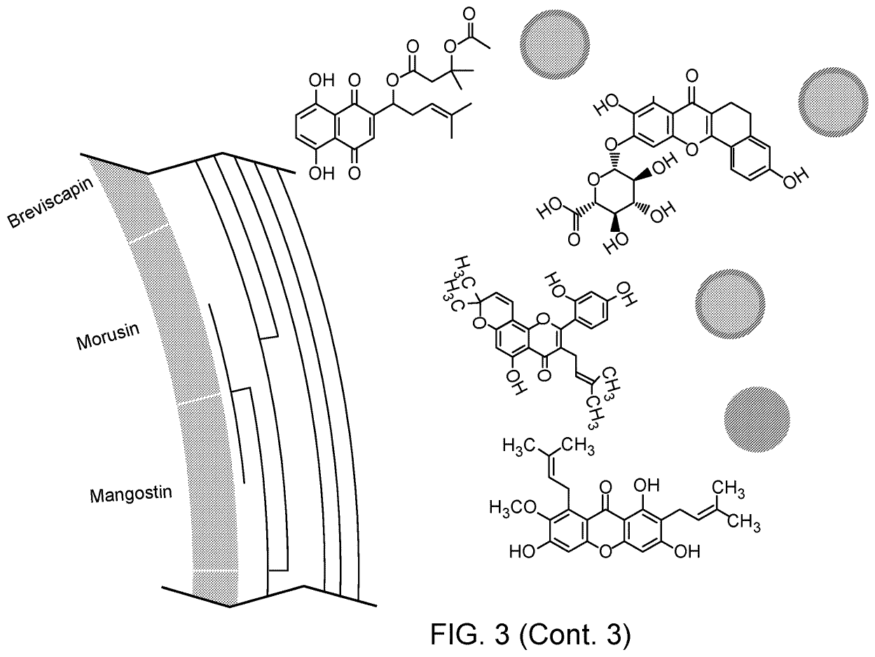

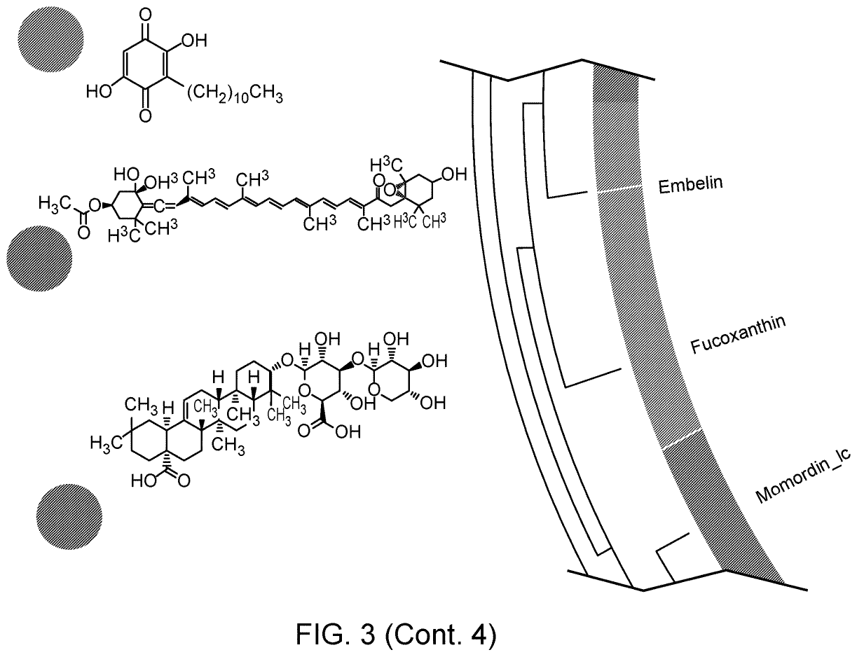

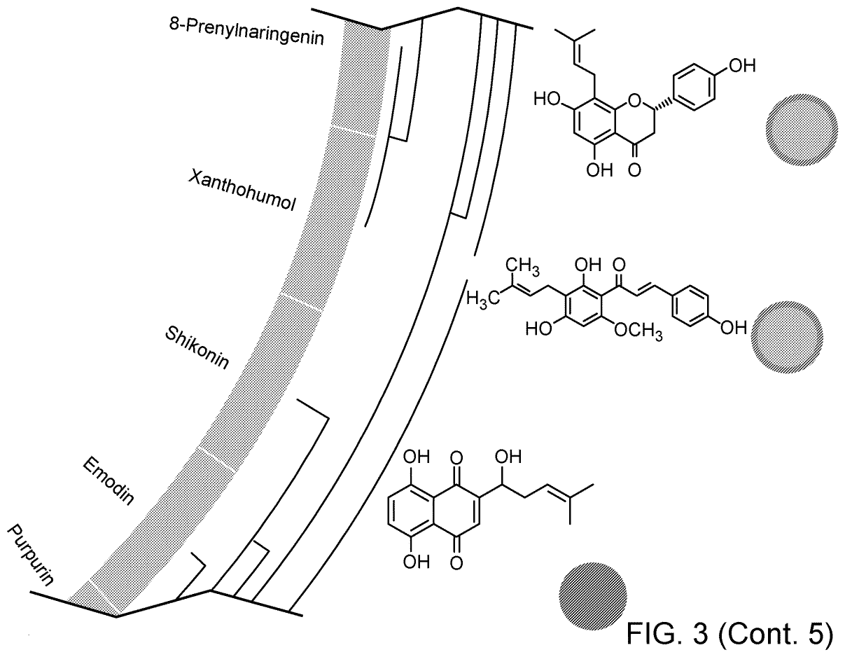

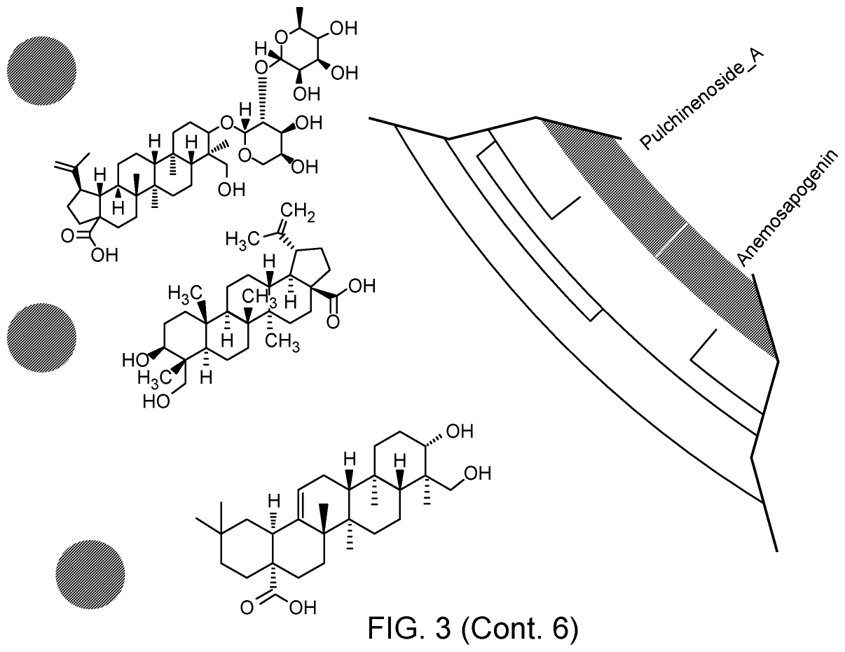

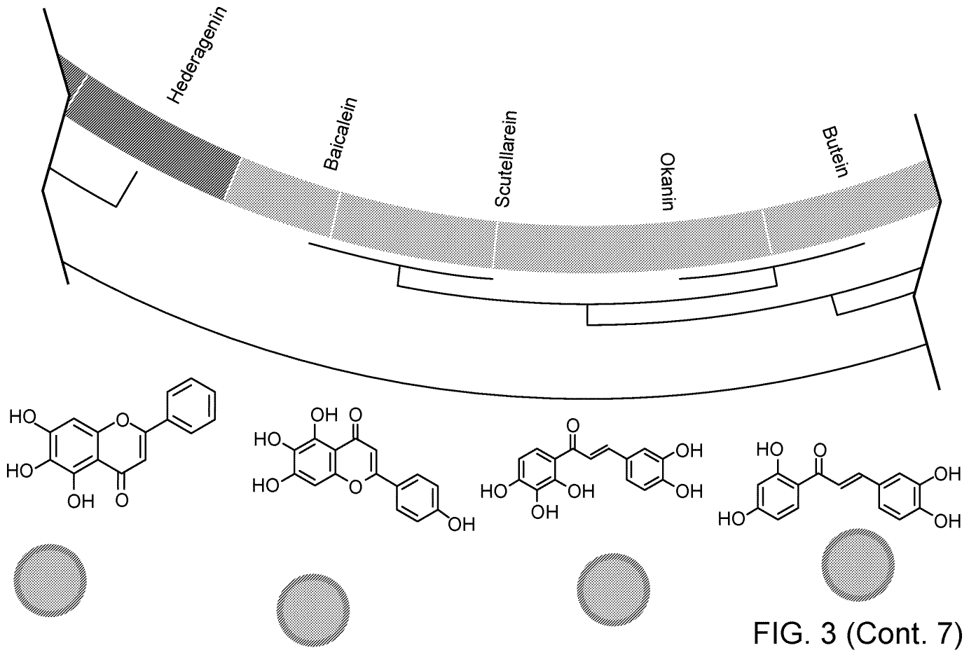

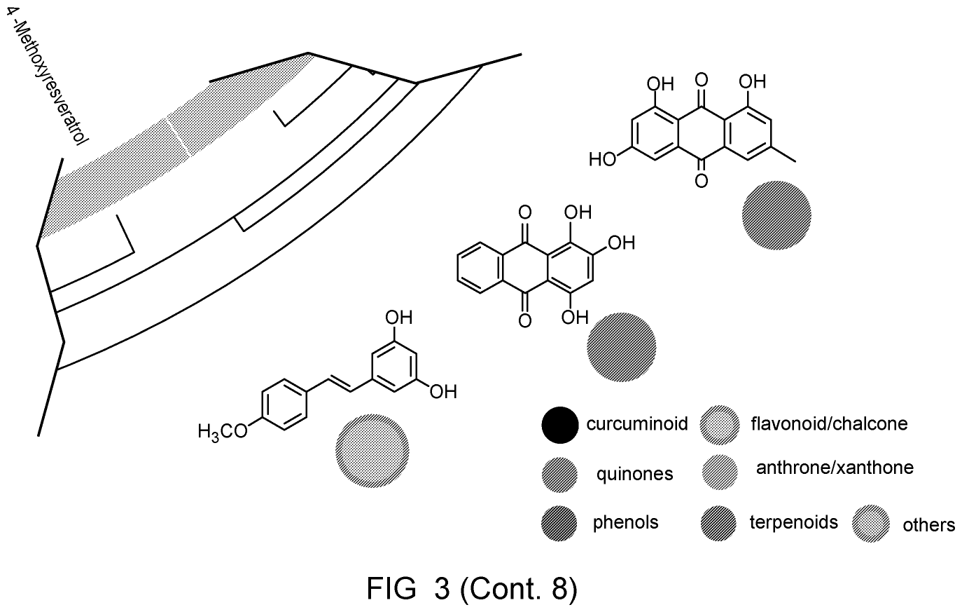

The chemical screening identified both positive and negative regulators of biofilm formation (e.g., biofilm comprising Gluconacetobacter diazotrophicus) (FIG. 2). The heat map was generated by the mean value of four biological replicates for each compound using the MORPHEUS software. For biofilm inducers, 7.36% (206 out of 2800) compounds increased biofilm formation more than 1.5 times compared to the DMSO control, and 2.75% (77 out of 2800) compounds increased biofilm formation more than 2 times compared to the DMSO control. 1.21% (34 out of 2800) compounds repressed biofilm formation to less than 50% compared to the DMSO control. More importantly, the top compounds identified showed greater induction on biofilm formation than that the flavonoid compound apigenin (Table 1), suggesting that they are more potent in protecting bacterial nitrogenase enzyme and increasing biological nitrogen fixation. 33.3% (10 out of 30) of the top biofilm inducers identified belong to the flavonoid/chlorone family, further confirming the important role of flavonoid in biofilm formation of nitrogen-fixing bacteria and biological nitrogen fixation. Other biofilm inducers identified belong to families such as curcuminoids, terpenoids, quinones, and anthrones/xanthones (FIG. 3).

TABLE 1

| | | | biofilm |
|---|---|---|---|
| Name | CAS | Category | induction |
| Breviscapin | 116122-35-2 | Flavonoid | 8.05 |
| Pulchinenoside A | 129724-84-1 | Triterpenoids | 7.62 |
| Sennoside A | 81-27-6 | Glycosides | 5.01 |
| Momordin Ic | 96990-18-0 | Triterpenoids | 4.98 |
| Butein | 487-52-5 | Others | 4.73 |
| Tannic acid | 1401-55-4 | Phenols | 4.32 |
| Curcumin | 458-37-7 | Curcuminoid | 4.27 |
| demethoxycurcumin | 24939-17-1 | Curcuminoid | 3.52 |
| Morusin | 62596-29-6 | Flavonoid | 3.49 |
| 1,2,3,4,6-O- | 14037-35-7 | Phenols | 3.39 |
| Pentagalloylglucose | | | |
| Punicalagin | 65995-63-3 | Phenols | 3.34 |
| Baicalein | 491-67-8 | Flavonoid | 3.33 |
| Bisdemethoxycurcumin | 24939-16-0 | Curcuminoid | 3.3 |
| Emodin | 518-82-1 | Anthraquinones | 3.26 |
| Fucoxanthin | 3351-86-8 | terpenoid | 3.2 |
| Embelln | 550-24-3 | Quinones | 3.11 |
| Agaric acid | 666-99-9 | Others | 3.03 |
| Dryocrassin ABBA | 12777-70-7 | Phenols | 3.02 |
| (±)-Shikonin | 54952-43-1 | Anthraquinones | 3.01 |
| Hederagenin | 465-99-0 | Triterpenoid | 2.93 |
| | | Glycosides | |
| Scutellarein | 529-53-3 | Flavonoid | 2.9 |
| Purpruin | 81-54-9 | Anthraquinones | 2.9 |
| Senagenin | 2469-34-3 | Triterpenoids | 2.85 |
| Protohyperioin | 648-03-8 | Quinones | 2.85 |
| Anemosapogerin | 85999-40-2 | Triterpenoids | 2.81 |
| Okanin | 484-75-4 | Chalcones | 2.78 |
| 8-Prenylnaringenin | 53846-50-7 | Flavonoids | 2.78 |
| Acetoxyisovalerylalkannin | 69091-17-4 | Quinones | 2.74 |
| Xanthohumol | 6754-56-1 | Chalcones | 2.65 |
| 4′-Methoxyresveratrol | 33626-08-3 | stilbenes | 2.6 |
| Genistein | 446-72-0 | Flavonoids | 2.03 |
| (+)-γ-Tocopherol | 54-28-4 | Vitamin E | 2.01 |
| Apigenin | 520-36-5 | Flavonoids | 1.72 |

The overrepresentation of the phenol group in biofilm inducers suggests that the phenol unit is the common backbone structure (FIG. 3). Table 2 shows natural compounds that induced biofilm formation (e.g., biofilm comprising Gluconacetobacter diazotrophicus) (as compared to the effects of DMSO). The cost of some of the identified compounds is very low. For example, tannic acid increased biofilm formation by 4.3-fold and its cost is 0.04% of the cost of apigenin (Table 2); curcumin increased biofilm formation by 4.3-fold and its cost is 0.2% of the cost of apigenin.

TABLE 2

| | | | Biofilm | | |
|---|---|---|---|---|---|
| Name | Source | Category | induction fold | price/g | price/mol |
| Tannic acid | Plant | Phenols | 4.32 | $0.20 | $341.94 |
| Curcumin | Plant | Ketone | 4.27 | $4.82 | $1,775.64 |
| Lumazine | Animal | Others | 1.76 | $16.40 | $2,691.57 |
| Indole-3-carbinol | Plant | Alkaloids | 1.59 | $10 | $1,471.70 |
| 3,4-Dihydroxyphenylacetic acid | Animal | Phenols | 1.55 | $36.60 | $6,154.29 |
| 6-Hydroxypurine | Organism | Alkaloids | 1.55 | $2.24 | $304.89 |
| Zinc Orotate | Organism | Others | 1.54 | $2.26 | $930.22 |
| 5-Hydroxy-1-tetralone | Plant | Others | 1.50 | $30.60 | $4,963.01 |
| PHYTOL | Plant | Diterpenoids | 1.48 | $0.39 | $114.46 |
| Quinic acid | Plant | Miscellaneous | 1.46 | $2.11 | $405.48 |
| Perillen | Plant | Alkaloids | 1.41 | $33.80 | $5,077.44 |
| Heptadecanoic acid | Animal | Fatty Acids | 1.40 | $7.16 | $1,936.42 |
| Pyrimidine | Animal | Diazines | 1.38 | $20 | $1,601.80 |
| α-D-Glucose 1-phosphate | Animal | Organooxygen | 1.33 | $12 | $3,865.32 |
| disodium salt | | compounds | | | |
| Apigenin | Plant | Flavonoids | 1.72 | $3,320 | $897,196.80 |

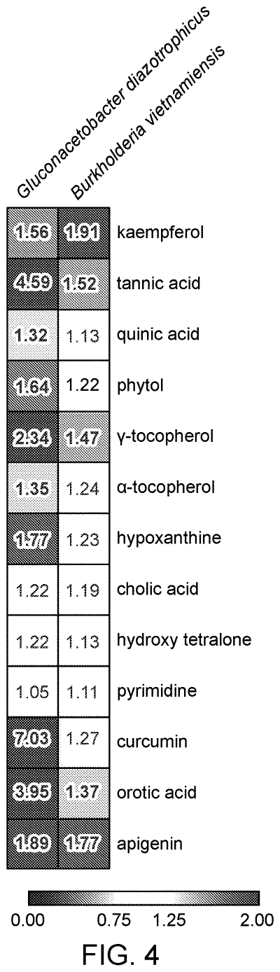

The effects on biofilm formation of some of these compounds were tested on two well-known nitrogen-fixing bacteria: Gluconacetobacter diazotrophicus and Burkholderia vietnamiensis. Kaempferol, tannic acid, phytol, γ-tocopherol, α-tocopherol, curcumin, orotic acid showed a promotion effect in both types of nitrogen-fixing bacteria tested (FIG. 4). The soil nitrogen-fixing bacteria was isolated as follows: root segments (5-10 cm below ground) were harvested from 16-week-old rice plants grown in the 22.5 ppm nitrogen regime (nitrogen-limiting conditions). Three individual roots were combined and ground up in a mortar and pestle after removing attached soil particles by vortexing. The tissues were filtered through 2 layers of cheesecloth and dissolved in 50 ml sterile water. A 10-4 dilution of the original solution was plated in Jensen's nitrogen-free medium containing 1.5% agar and cultured for 7 days at 28 °C. Individual colonies were picked up and grown on fresh Jensen's nitrogen-free medium for a secondary selection. Eighty random colonies that survived the second selection were tested for their biofilm formation in the presence of apigenin, tannic acid, or curcumin.

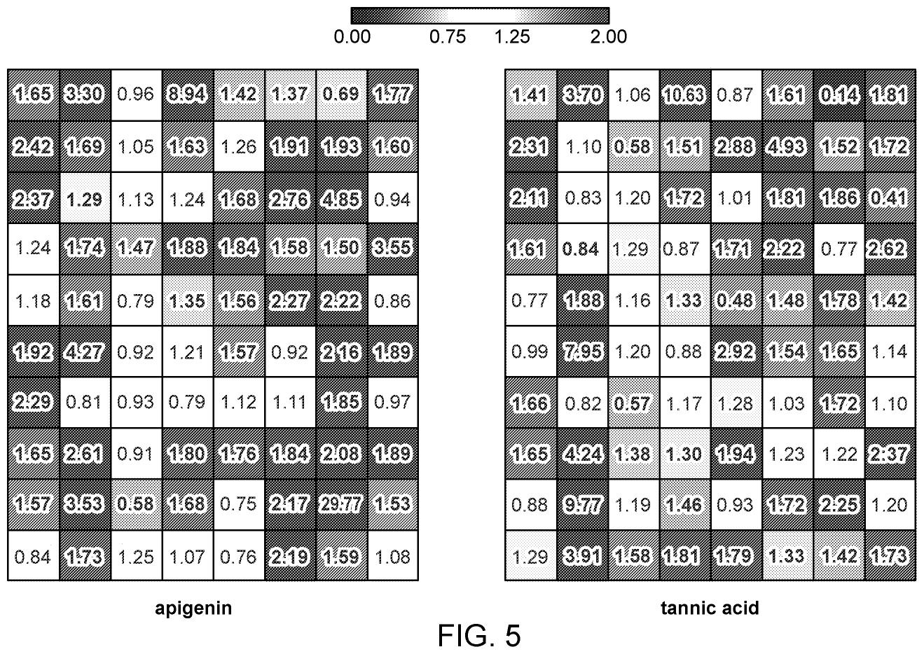

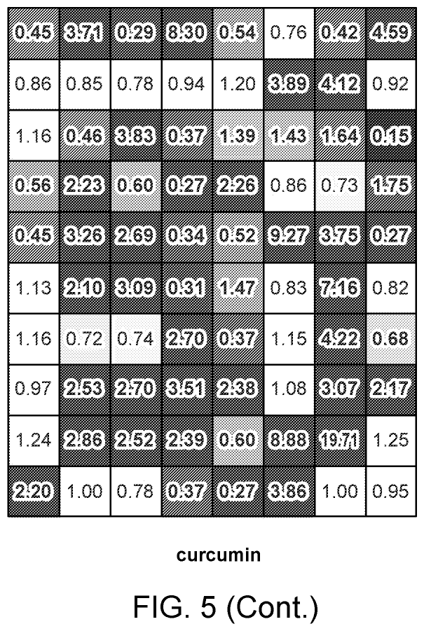

As shown in FIG. 5, apigenin and tannic acid were biofilm inducers for various nitrogen-fixing bacteria. Apigenin promoted biofilm at least 20% more than the DMSO control in 71.2% (57 out of 80) of the nitrogen-fixing bacteria tested. Tannic acid showed positive effect on 68.8% (55 out of 80) of the bacteria tested. Curcumin showed a narrower effect on the biofilm induction as 47.5% (38 out of 80) of the nitrogen-fixing bacteria produced more biofilm.

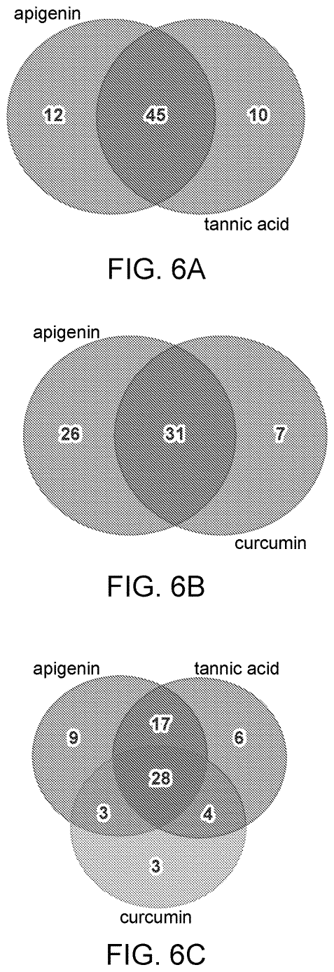

The Veen diagram showed the overlap of soil diazotrophs whose biofilm can be induced by both apigenin and tannic acid in FIG. 6A. Among the bacteria whose biofilm can be induced by apigenin, 78.9% (45 out of 57) can also be induced by tannic acid. Among the bacteria whose biofilm can be induced by tannic acid, 81.8% (45 out of 55) can also be induced by apigenin. Among the bacteria whose biofilm can be induced by apigenin, 54.4% (31 out of 57) can also be induced by curcumin (FIG. 6B). Among the bacteria whose biofilm can be induced by tannic acid, 81.6% (31 out of 38) can also be induced by apigenin. Giving that various soil diazotrophs acted similarly to both tannic acid and apigenin when forming biofilms, tannic acid would be a cheaper alternative to apigenin in seed treatments (e.g., seed coating) to increase biological nitrogen fixation. Tannic acid has been used as a safe and environment friendly food additive and is included in the European Union list of food flavorings. In agricultural practices, tannic acid could be used as a substitute for safer fungicides against fungus such as Fusarium graminearum (Forrer et al., 2014) and Penicillium digitatum (Zhu et al., 2019). Similarly to apigenin, tannic acid is well known for its antimicrobial activity against pathogens (Ekambaram et al., 2016).

Effect of Chemicals on Triticum aestivum

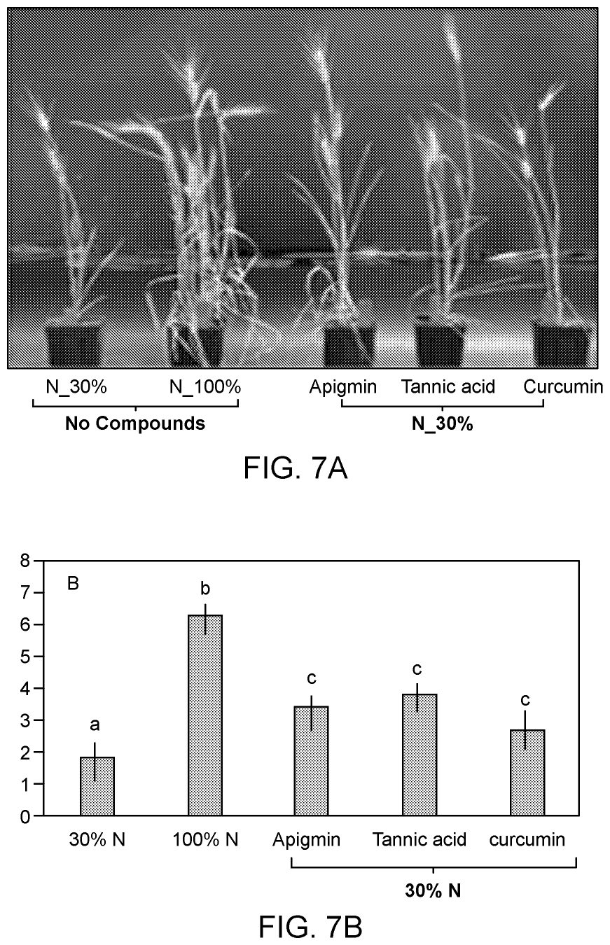

Seeds of a hexaploid wheat (Triticum aestivum) were germinated in the lab and transferred to pots containing commercial soil. Seeds were grown in two different nitrogen concentrations. Seeds grown under 100% N₂ were provided with solutions containing 140 ppm N₂, while seeds grown under 30% N₂ were provided with solutions containing 42 ppm N₂. As indicated in FIGS. 7A and 7B, 2 mL of a solution containing 100 μM apigenin, tannic acid, or curcumin was added to the soil one week after seedlings were transplanted to pots containing soil.

### REFERENCES

1. Ekambaram, S. P., Perumal, S. S., and Balakrishnan, A. (2016) Scope of Hydrolysable Tannins as Possible Antimicrobial Agent. Phyther. Res., 30, 1035-1045.
2. Forrer, H. R., Musa, T., Schwab, F., Jenny, E., Bucheli, T. D., Wettstein, F. E., and Vogelgsang, S. (2014) Fusarium head blight control and prevention of mycotoxin contamination in wheat with botanicals and tannic acid. Toxins (Basel)., 6, 830-849.
3. Zhu, C., Lei, M., Andargie, M., Zeng, J., and Li, J. (2019) Antifungal activity and mechanism of action of tannic acid against Penicillium digitatum. Physiol. Mol. Plant Pathol., 107, 46-50.
4. Yan, D., Tajima, H., Cline, L. C., Fong, R. Y., Ottaviani, J. I., Shapiro, H. Y., Blumwald, E. (2022). Genetic modification of flavone biosynthesis in rice enhances biofilm formation of soil diazotrophic bacteria and biological nitrogen fixation.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A composition comprising a crop plant or seeds of a crop plant treated with a compound of Table 1 in an amount sufficient to induce biofilm formation, wherein the biofilm comprises nitrogen-fixing bacteria.

2. The composition of claim 1, wherein the crop plant is a seedling.

3. The composition of any one of claims 1 to 2, wherein the roots of the crop plant is treated with the compound of Table 1.

4. The composition of claim 1, wherein the compound is tannic acid.

5. The composition of any one of claims 1 to 4, wherein the crop plant is a cereal crop.

6. The composition of any one of claims 1 to 5, wherein the crop plant is selected from the group consisting of corn, wheat, rice, soy, cotton, canola, and sugarcane.

7. The composition of claim 6, wherein the crop plant is rice.

8. A method of producing a crop plant or seeds of the crop plant that induce biofilm formation, the method comprises treating the crop plant or the seeds with a compound of Table 1, wherein the biofilm comprises nitrogen-fixing bacteria.

9. The method of claim 8, wherein the method comprises treating the seeds with the compound of Table 1 and the treating is performed prior to planting the seeds in a soil comprising the nitrogen-fixing bacteria.

10. The method of claim 9, further comprising planting the treated seeds in the soil.

11. The method of claim 8, wherein the method comprises treating a crop plant with the compound of Table 1.

12. The method of claim 11, wherein the crop plant is a seedling and the treating is performed prior to planting the seedling in a soil comprising the nitrogen-fixing bacteria.

13. The method of claim 11, wherein the crop plant is a seedling and the treating is performed after planting the seedling in a soil comprising the nitrogen-fixing bacteria.

14. The method of claim 12, further comprising planting the treated seedling in the soil.

15. The method of any one of claims 8 to 14, wherein the compound is tannic acid.

16. The method of any one of claims 8 to 15, wherein the seeds are treated with the compound by seed coating or seed injection.

17. The method of claim 16, wherein the seed coating is selected from the group consisting of seed dressing, film coating, pelleting, and encrusting.

18. The method of any one of claims 8 to 17, wherein the crop plant is a cereal crop.

19. The method of any one of claims 8 to 18, wherein the crop plant is selected from the group consisting of corn, wheat, rice, soy, cotton, canola, and sugarcane.

20. The method of claim 19, wherein the crop plant is rice.

21. The method of any one of claims 8 to 20, wherein the crop plant or the seeds are planted under reduced inorganic nitrogen conditions.

22. The method of claim 21, wherein the amount of inorganic nitrogen in a soil is less than 90%, 80%, 70%, 60% or 50% of the standard amount of nitrogen for the crop plant.

23. The method of any one of claims 8 to 22, wherein the nitrogen-fixing bacteria in a soil in which the crop plant or the seeds are grown show greater biofilm formation than control nitrogen-fixing bacteria in a soil in which a control plant or control seeds not treated with the compound are grown.

24. The method of any one of claims 8 to 23, wherein the crop plant grown in a soil comprising the reduced amount of inorganic nitrogen assimilates at least twice the amount of atmospheric nitrogen than the amount assimilated by a control plant grown in equivalent soil, and wherein the control plant are not treated with the compound.

25. The method of any one of claims 8 to 24, wherein the crop plant or seeds of the crop plant induces a larger area of biofilm than a biofilm induced by a control crop plant or control seeds not treated with the compound of Table 1.

26. The method of any one of claims 8 to 25, wherein the biofilm contains a greater quantity of nitrogen-fixing bacteria than a biofilm induced by a control crop plant or control seeds not treated with the compound of Table 1.

27. The method of any one of claims 8 to 26, wherein the biofilm produces more fixed nitrogen than a biofilm induced by a control crop plant or control seeds not treated with the compound of Table 1.

28. A method for selecting for a compound that induces biofilm formation, the method comprises:
1) treating the crop plant or the seeds with a compound of Table 1;
2) comparing the amount of biofilm formation in the soil in which the crop plant or the seeds are grown with the amount of biofilm formation in the soil in which a control plant or control seeds not treated with a compound of Table 1 are grown;
3) selecting a compound for the crop plant or the seeds that induces more biofilm formation compared to that of the control plant or control seeds,
wherein the biofilm comprises nitrogen-fixing bacteria.

29. The method of claim 28, wherein in step 3), the biofilm induced by compound contains a greater quantity of nitrogen-fixing bacteria than that of the biofilm in the soil of the control crop plant or control seeds not treated with the compound of Table 1.

30. A method for selecting for a crop plant or seeds of the crop plant that induce greater nitrogen assimilation relative to a control plant or control seeds, the method comprises:
1) treating the crop plant or the seeds with a compound of Table 1;
2) comparing the amount of nitrogen assimilated into the crop plant or the seeds with the amount of nitrogen assimilated into the control crop plant or control seeds not treated with a compound of Table 1;
3) selecting for the crop plant or the seeds that have greater nitrogen assimilation compared to that of the control plant or control seeds.

31. The method of claim 30, wherein the crop plant or the seeds have at least 0.1-fold greater amount of nitrogen assimilated than the amount of nitrogen assimilated into the control crop plant or control seeds not treated with the compound of Table 1.

32. A method for selecting for a crop plant that have a greater number of tillers, tassels, and/or spikes, and/or greater seed yield relative to a control plant, the method comprises:
1) treating the crop plant with a compound of Table 1;
2) comparing the number of tillers, tassels, and/or spikes, and/or amount of seed yield with that of the control crop plant not treated with a compound of Table 1;
3) selecting for the crop plant that has the greater number of tillers, tassels, and/or spikes, and/or greater amount of seed yield compared to that of the control plant.

33. The method of claim 32, wherein the crop plant has at least 5% more tillers, tassels, and/or spikes, and/or seed yield than that of the control crop plant not treated with the compound of Table 1.

34. The method of any one of claims 28 to 33, wherein the crop plants or seeds are grown under low nitrogen condition.

35. The method of any one of claims 28 to 34, wherein the crop plant is selected from the group consisting of corn, wheat, rice, soy, cotton, canola, and sugarcane.
